(19) United States
(12) Patent Application Publication
(10) Pub. No.: US 2008/0233634 A1
(43) Pub. Date: Sep. 25, 2008

(54) NUCLEIC ACID DETECTION DEVICE

(76) Inventors: Jun OKADA, Tokyo (JP); Sadato Hongo, Yokohama-shi (JP); Keiko Ito, Kawasaki-shi (JP); Kenji Ooki, Yokohama-shi (JP); Nobuhiro Gemma, Yokohama-shi (JP)

Correspondence Address: OBLON, SPIVAK, MCCLELLAND MAIER & NEUSTADT, P.C., 1940 DUKE STREET, ALEXANDRIA, VA, 22314, US

(21) Appl. No.: 12/048,690

(22) Filed: Mar. 14, 2008

(30) Foreign Application Priority Data
Mar. 23, 2007 (JP) 2007-077876

(51) Int. Cl. C12M 1/00

(52) U.S. Cl. 435/287.2

(57) ABSTRACT

A nucleic acid detection device is provided with a closed channel formed of a first channel portion, through which a washing reagent storage section for storing a washing reagent for washing a detection section for nucleic acid detection communicates with the detection section, and a second channel portion through which a pretreatment section for nucleic acid treatment communicates with the detection section. The closed channel is connected with a gas inlet/outlet path for communication with the outside. The gas inlet/outlet path is blocked by a sealing mechanism before nucleic acid detection. In storing a pretreatment reagent and the washing reagent frozen, the gas inlet/outlet path is kept open and connected to the channel. Thus, there is provided a nucleic acid detection device having a structure for preventing leakage of nucleic acid samples to the outside and which can be stored for a long period of time with the reagents therein.

### CROSS-REFERENCE TO RELATED APPLICATIONS

This application is based upon and claims the benefit of priority from prior Japanese Patent Application No. 2007-077876, filed Mar. 23, 2007, the entire contents of which are incorporated herein by reference.

### BACKGROUND OF THE INVENTION

1. Field of the Invention

The present invention relates to a nucleic acid detection device, and more particularly, to a nucleic acid detection device for fully automatically performing nucleic acid detection and processes for pretreatment therefor, thereby detecting a target nucleic acid in each sample.

2. Description of the Related Art

With the recent developments in genetic engineering, disease diagnosis or prevention based on genetic data has become feasible in the medical field. This diagnosis is called genetic diagnosis, whereby human genetic defects or changes that may cause diseases can be detected to serve as data for the diagnosis or prediction of the diseases in their precritical or very early stages. As the human genome has been decoded, moreover, investigations on the association between the genotype and the incidence of plague have been advanced, and a therapy customized for each individual's genotype (tailor-made medicine) is becoming practicable. Thus, it is very important to detect genes and determine the genotype with ease.

A conventionally known system for detecting nucleic acid uses several devices, such as a nucleic acid extractor, nucleic acid amplifier, hybridizer, nucleic acid detector, data analyzer, etc. In the system of this type, preparation of other samples than those realized by these devices, and movement of samples between the devices, etc., requires substantial manpower.

In recent years, devices that automatically perform processes from a hybridization reaction to data analysis have been developed. Further, a fully automated nucleic acid detector has recently been developed that can automatically perform processes from nucleic acid extraction to data analysis.

The PCR or LAMP method is mainly used for nucleic acid amplification in the nucleic acid detector or detection system described above. This method provides a very high amplification rate. If the smallest amount of a different nucleic acid is mixed into an unamplified sample, however, the high amplification rate causes the mixed nucleic acid to be over-amplified and therefore entails erroneous detection. It is known that nucleic acid molecules are stable even in a dry state and adsorbable to various substances, and that they sometimes may be suspended in air. In order to prevent erroneous detection, therefore, the site of nucleic acid extraction requires a strict management system that prohibits introduction of amplified samples, for example.

A sealed device disclosed in JP-A 2005-261298 (KOKAI) is proposed as a device for solving the problems of mixing-in of undetectable nucleic acid molecules and leakage of nucleic acid samples to the outside.

In this sealed device, a plurality of reaction processes are automatically controlled for several hours, so that very small amounts of reagents of a plurality of types, e.g., in nanoliters or microliters, are used in extraction and amplification processes, in particular, and their control is believed to be very difficult.

If practical use is taken into consideration, moreover, prolonged storage is essential for a nucleic acid detection device that contains reagents of a plurality of types. Heretofore, however, no examination has been made on the prolonged storage of the nucleic acid detection device with very small amounts of reagents therein. While frozen storage is an effective method for storing the various reagents stably without degradation, it is essential to remove various obstructions that are attributable to freezing of the reagents in the device. In a structure such that the reagents are stored in the middle of a channel, for example, gas volumes in channel portions before and behind the reagents are reduced according to equation (1) if the temperature drops. As the pressure is reduced, the reagents are pulled and moved inevitably.

P×V=n×R×T. (1)

If the temperature further drops in the moved state, the reagents are frozen in positions to which they are moved, and fail to be completely restored to their original positions even when they are thawed thereafter. If the reagents are very small in amount, in particular, they cannot be controlled with ease. When the reagents move, their components adsorb to the wall surface of the channel, and residual liquids, if any, result in a fatal defect. Another problem is that each reagent evaporates very slowly even in a frozen state. If the vicinity of a storage section for a very small amount of a pretreatment reagent is open to the outside of the device, therefore, the reagent is considerably influenced by the evaporation even when it is stored frozen, thus entailing a fatal defect.

As mentioned before, a major object of the development of a fully automated nucleic acid analyzer is to develop a device that has a structure for preventing mixing-in of undetectable nucleic acid molecules and leakage of nucleic acid samples to the outside and can be stored for a long period of time with various reagents contained therein.

### BRIEF SUMMARY OF THE INVENTION

According to an aspect of this invention, there is provided a nucleic acid detection device comprising:

a pretreatment section configured to treat a nucleic acid contained in a specimen before nucleic acid detection;

a detection section configured to detect the pretreated nucleic acid;

a pretreatment reagent storage section which communicates with the pretreatment section and stores a pretreatment reagent to be used for the treatment in the pretreatment section;

a washing reagent storage section configured to store a washing reagent which is used to wash the detection section after the specimen is supplied from the pretreatment section to the detection section;

a channel including a first channel portion through which the washing reagent storage section communicates with the detection section and a second channel portion through which the pretreatment section communicates with the detection section, the first and second channel portions forming a closed channel;

a gas inlet/outlet path through which the closed channel communicates with the outside; and

a sealing mechanism which blocks the gas inlet/outlet path and keeps the channel closed before the nucleic acid detection, the sealing mechanism being configured to keep the gas inlet/outlet path and the channel open when the pretreatment reagent and the washing reagent are stored frozen and externally block the gas inlet/outlet path after the pretreatment reagent and the washing reagent are thawed.

According to another aspect of the invention, there is provided a nucleic acid detection device comprising:

a pretreatment section configured to treat a nucleic acid contained in a specimen before nucleic acid detection;

a detection section configured to detect the nucleic acid;

a pretreatment reagent storage section which communicates with the pretreatment section and stores a pretreatment reagent to be used for the treatment in the pretreatment section;

a washing reagent storage section configured to store a washing reagent which is used to wash the detection section after the specimen is supplied from the pretreatment section to the detection section;

a channel including a first channel portion through which the washing reagent storage section communicates with the detection section and a second channel portion through which the pretreatment section communicates with the detection section, the first and second channel portions forming a closed channel; and

a fastener which blocks a part of a channel in the pretreatment section from the channel, thereby isolating the part of the channel in the pretreatment section, the fastener having an opening function to cause the pretreatment section to communicate with the channel during the nucleic acid detection.

According to a further aspect of the invention, there is provided a nucleic acid detector for nucleic acid detection, which incorporates therein a nucleic acid detection device comprising a pretreatment section configured to treat a nucleic acid contained in a specimen before nucleic acid detection, a detection section configured to detect the nucleic acid, a pretreatment reagent storage section which communicates with the pretreatment section and stores a pretreatment reagent to be used for the treatment in the pretreatment section, a washing reagent storage section configured to store a washing reagent which is used to wash the detection section after the specimen is supplied from the pretreatment section to the detection section, and a channel composed of a first channel portion through which the washing reagent storage section communicates with the detection section and a second channel portion through which the pretreatment section communicates with the detection section, the first and second channel portions forming a closed channel, the nucleic acid detector comprising a heating section which blocks the pretreatment section from the channel during the nucleic acid detection and heats the pretreatment section.

### BRIEF DESCRIPTION OF THE SEVERAL VIEWS OF THE DRAWING

FIG. 1 is a perspective view schematically showing a nucleic acid detection device according to one embodiment of the invention;

FIG. 2 is a perspective view schematically showing the detection device of FIG. 1;

FIGS. 3A, 3B, 3C and 3D are a top view, a front view, and opposite side views, respectively, showing developments of the detection device of FIG. 1;

FIGS. 4A and 4B are a top view and a side view, respectively, schematically showing a side structure of the detection device of FIGS. 1 and 2;

FIGS. 5A and 5B are side views schematically showing another side structure of the detection device of FIGS. 1 and 2;

FIG. 6 is a block diagram typically showing a nucleic acid detector that utilizes the nucleic acid detection device according to the one embodiment of the invention;

FIG. 7 is a flowchart showing processes from sample injection to nucleic acid detection in the detector of FIG. 6;

FIG. 8 is a diagram showing a typical layout of channels and gas inlet/outlet paths of the detection device of FIGS. 1 and 2;

FIG. 9 is a diagram showing another typical layout of the channels and gas inlet/outlet paths of the detection device of FIGS. 1 and 2;

FIG. 10 is a diagram showing another typical layout of the channels and gas inlet/outlet paths of the detection device of FIGS. 1 and 2;

FIG. 11 is a diagram showing an alternative layout of the channels and gas inlet/outlet paths of the detection device of FIGS. 1 and 2;

FIGS. 12A and 12B are diagrams showing another layout of the channels and gas inlet/outlet paths of the detection device of FIGS. 1 and 2;

FIGS. 13A and 13B are diagrams showing a structure example of the gas inlet/outlet path of the detection device of FIGS. 1 and 2;

FIGS. 14A and 14B are diagrams showing another structure example of the gas inlet/outlet path of the detection device of FIGS. 1 and 2;

FIGS. 15A and 15B are diagrams showing a further structure example of the gas inlet/outlet path of the detection device of FIGS. 1 and 2;

FIGS. 16A and 16B are diagrams showing an alternative structure example of the gas inlet/outlet path of the detection device of FIGS. 1 and 2;

FIGS. 17A, 17B and 17C are diagrams showing another structure example of the gas inlet/outlet path of the detection device of FIGS. 1 and 2;

FIGS. 18A, 18B, 18C and 18D are diagrams showing an example of a structure in which nucleic acid extraction/amplification chambers of the detection device of FIGS. 1 and 2 are sealed by a clip; and

FIGS. 19A, 19B, 19C and 19D are diagrams showing a structure example of a heating section for heating the nucleic acid extraction/amplification chambers of the detection device of FIGS. 1 and 2.

### DETAILED DESCRIPTION OF THE INVENTION

A nucleic acid detection device according to one embodiment of the invention will now be described with reference to the accompanying drawings as required.

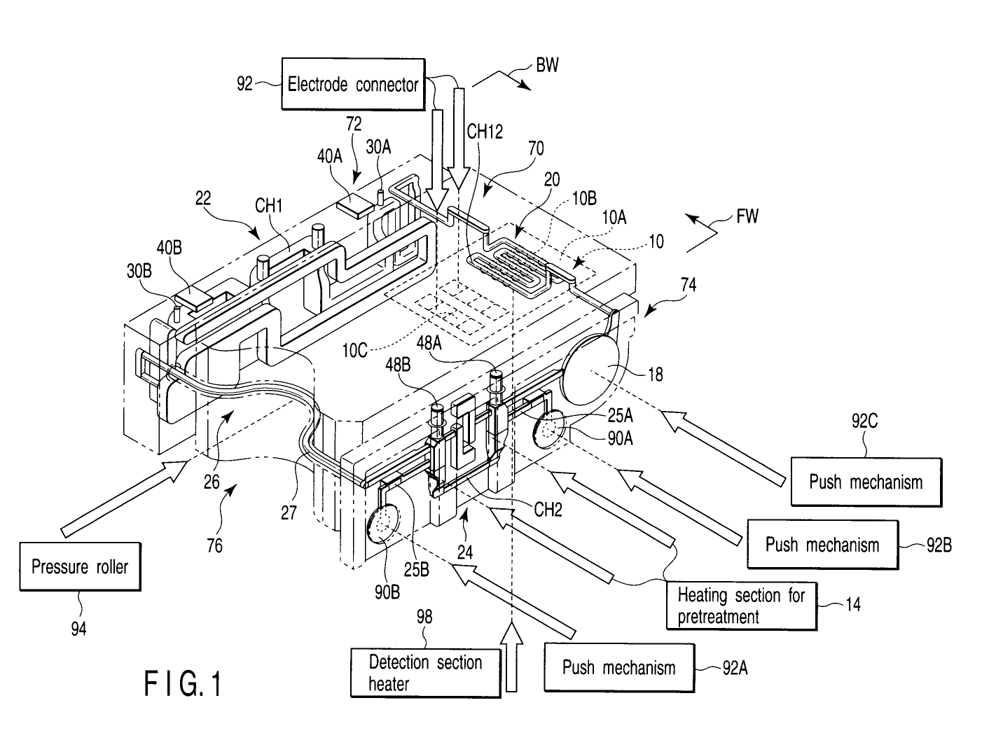

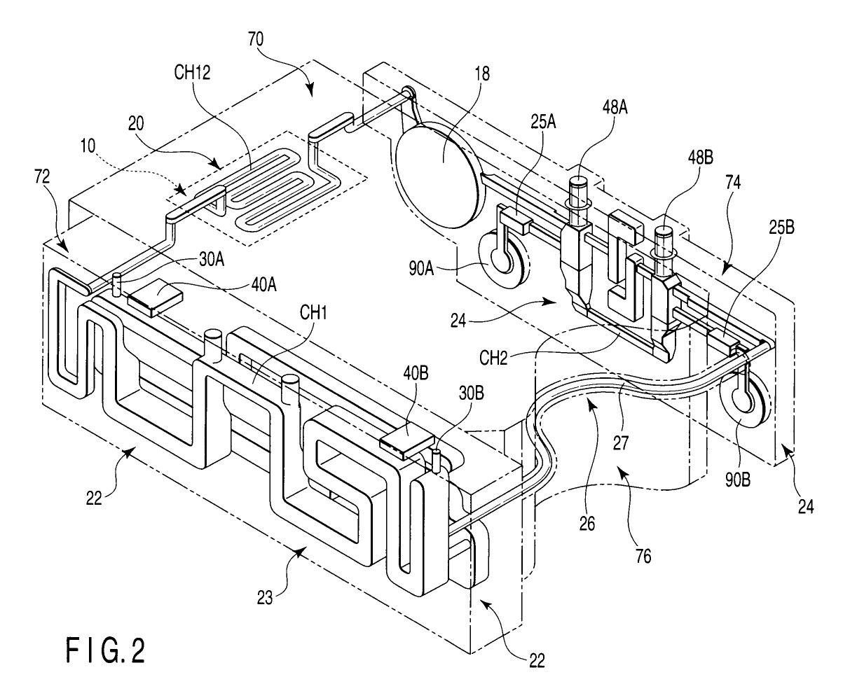

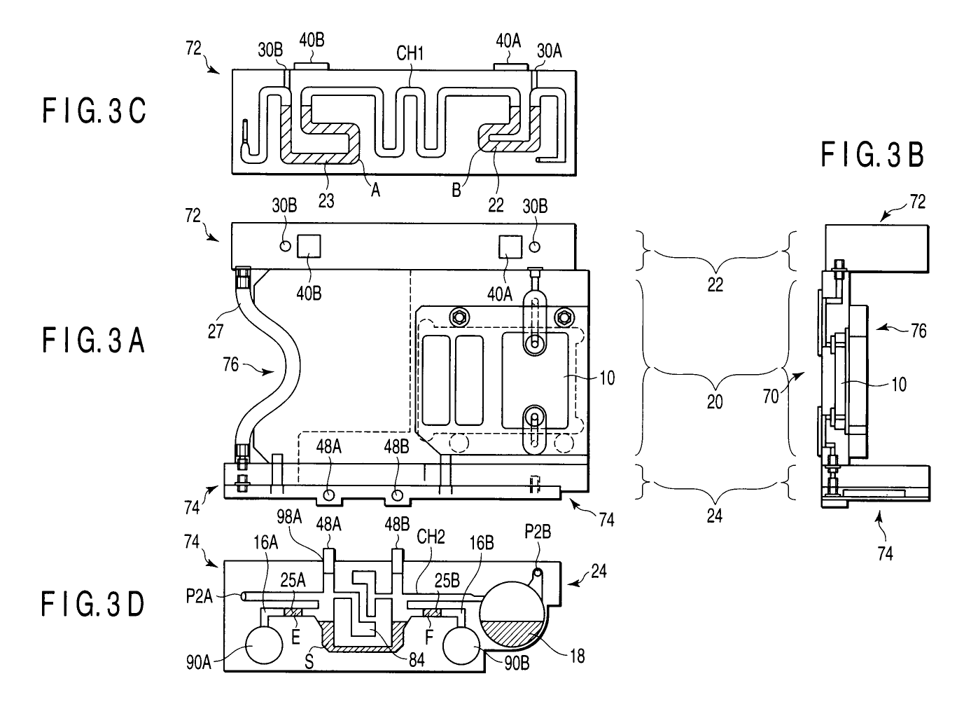

FIGS. 1 and 2 are perspective views schematically showing the nucleic acid detection device according to the embodiment of the invention, and FIGS. 3A to 3D are developments showing the detection device of FIGS. 1 and 2.

As shown in FIGS. 1 to 3D, the nucleic acid detection device, which is also called as a nucleic acid detection cassette, is composed of a top structure 70 and side structures 72 and 74 provided individually on the opposite sides of the top structure 70. As a whole, the detection device is substantially in the form of a saddle. A base structure 76 is interposed between the side structures 72 and 74, and all these structures are unified. The top structure 70 is provided with a detection section 20 for detecting nucleic acid so as to communicate with channels CH1 and CH2. The one side structure 74 is provided with a pretreatment section 24 and pretreatment reagent storage sections 25A and 25B. The pretreatment section 24 contains a pretreatment reagent S and communicates with the channel CH1. The storage sections 25A and 25B communicate with the channel CH1 and store pretreatment reagents E and F, respectively, which are used for treatment in the pretreatment section 24. The storage sections 25A and 25B individually communicate with the pretreatment section 24. The pretreatment section 24 performs a process for extracting nucleic acid from a applied reagent and/or a nucleic acid amplification process. The other side structure 72 is provided with a washing reagent storage section 22 and an intercalator solution storage section 23. The washing reagent storage section 22 stores a washing reagent B and communicates with the channel CH2. The intercalator solution storage section 23 stores an intercalator solution A, which interacts with the nucleic acid after a hybridization reaction, and communicates with the channel CH2. The storage sections 22 and 23 are separated by a gas in the channel CH2. Further, the base structure 76 is provided with a liquid feed section 26. The channels CH1 and CH2, liquid feed section 26, pretreatment section 24, detection section 20, and storage sections 22 and 23 are formed in a closed annular channel as a whole. Furthermore, the channel CH2 is provided with a reagent chamber 18, which stores a sample salt concentration adjustment reagent D. This reagent D may be doped with positive and negative control reagents, a hybridization-enhancing reagent, fluorescent dye label reagent, etc.

In the channel CH1, as shown in FIG. 3D, the liquid pretreatment reagent S stored in the pretreatment section 24 and the pretreatment reagents E and F stored in the pretreatment reagent storage sections 25A and 25B are spatially separated by the gas. Likewise, in the channel CH2, the liquid reagent A, e.g., an intercalator solution, and the reagent B, e.g., a washing reagent, are spatially separated by the gas. The specimen S and the reagents A and B, which are liquid, are moved in the annular channels CH1 and CH2 by a pump in the liquid feed section 26, e.g., a peristaltic pump formed of a pressure roller 94 and an elastic piping tube 27.

As shown in FIG. 1, the peristaltic pump is composed of the base structure 76 and the elastic piping tube 27. The base structure 76 has a curved surface such that the pressure roller 94 can travel thereon. The piping tube 27 is exposed along the curved surface. The roller 94 and the tube 27 constitute the pump for liquid feed. As the pressure roller 94 travels along the curved surface while pressuring the piping tube 27, the reagents A, B and S are fed in a forward direction FW or a backward direction BW. Thus, when the pressure roller 94 that is mounted on the body of the nucleic acid detection device is brought into contact with the elastic piping tube 27 and rotated, the tube 27 is deformed so that the gas or liquid therein is supplied squeezed. As seen from its structure, therefore, the pressure roller can be moved without directly contacting the liquid in a cassette channel. If the rotation direction of the pressure roller 94 is reversed, the direction of liquid feed is reversed. Further, the rotation of the roller 94 can be externally controlled to move the liquid in the tube. In the liquid feed section 26, therefore, the interior of the cassette never contacts the outside, so that the liquid in the cassette can be prevented from contaminating the ambience.

Furthermore, a DNA chip 10 is disposed in the detection section 20. Thus, the piping tube 27 of the pump is connected to a channel over the DNA chip 10 through the annular channels CH1 and CH2. The channels CH1 and CH2, a pump section (liquid feed section 26) between these channels, and the detection section 20 including the DNA chip 10 constitute a sealed channel that is closed against the outside. The channels CH1 and CH2 are defined having a sealed structure in the side structures 72 and 74 of the nucleic acid detection device. Likewise, the DNA chip 10 is set in the top structure 70 of the detection device. If a pump channel or the elastic piping tube 27 in the base structure 76 is externally operated, the specimen S and the reagents A and B are supplied to a detection region over the DNA chip 10.

In the present embodiment, the DNA chip 10 in the detection section 20 is of a current detection type such as to perform an electrochemical nucleic acid detection method using an intercalator. The DNA chip 10 is provided with a nucleic acid detection substrate for hybridization and nucleic acid detection reactions. Individual electrodes of Au or the like are arranged preferably in a matrix on the nucleic acid detection substrate, and nucleic acid probe DNAs are immobilized individually on the individual electrodes. Counter electrodes corresponding to the individual electrodes and reference electrodes are arranged on the DNA chip 10.

If pretreated specimens (DNA samples) are fed onto the DNA chip 10, a target DNA included in the specimens and the probe DNAs are subjected to a hybridization reaction. Then, unreacted DNAs on the DNA chip 10 are washed with the washing reagent B. Thereafter, the intercalator solution is fed onto the DNA chip 10, whereby the intercalator is caused to act on double chains of the target DNA and the probe DNAs. A voltage is applied between the counter electrodes and the individual electrodes, and the presence of the target DNA is determined by detecting current flows through the individual electrodes.

As shown in FIG. 1, a channel CH12 that is connected to the channels CH1 and CH2 are formed on a substrate 10A of the DNA chip 10. In order to make the channel CH12 communicate with the channels CH1 and CH2, the DNA chip 10 is provided with a port that is connected to the channels CH1 and CH2. Specifically, the channel CH12 has therein the DNA chip 10 on which electrodes, such as individual electrodes 10B, counter electrodes, and reference electrodes, are arranged, and the liquid is fed between the individual electrodes 10B and the other electrodes. Wires of the DNA chip 10 are connected individually to electrode pads 10C, extending from the individual electrodes 10B and the counter electrodes and the reference electrodes that are provided corresponding to the individual electrodes 10B on the substrate 10A. The electrode pads 10C are arranged so as to be exposed on the top structure 70. In detecting current, they are contacted or connected to an electrode connector 92 on the nucleic acid detection device side. Further, the channel CH12 is formed by joining a chip substrate and silicone rubber with a groove for use as a channel, for example.

A detailed description of the nucleic acid detection method of the current detection type using the intercalator is omitted herein. For the details of this nucleic acid detection method, refer to U.S. Pat. Nos. 5,776,672 and 5,972,692 (by Koji Hashimoto et al. and Assignee Kabushiki Kaisha Toshiba), filed Jul. 7, 1998 and Oct. 26, 1999, respectively, and Japanese Patent No. 2573443 corresponding thereto. The descriptions in the specifications of these U.S. patents constitute a part of this specification.

Preferably, a liquid sensor is located in the detection region of the DNA chip 10 so that it can detect the arrival of liquids, including the specimen S and the reagents A, B and D, and that the liquid feed operation of the pump can be controlled based on the resulting liquid detection output. More preferably, the liquid sensor should be able to identify the reagents A and B and the specimen S, detect the liquid feed, and determine the type of the fed liquid. As a pretreatment, moreover, the success or failure in the amplification should preferably be determined for the pretreated reagents having undergone the nucleic acid amplification process.

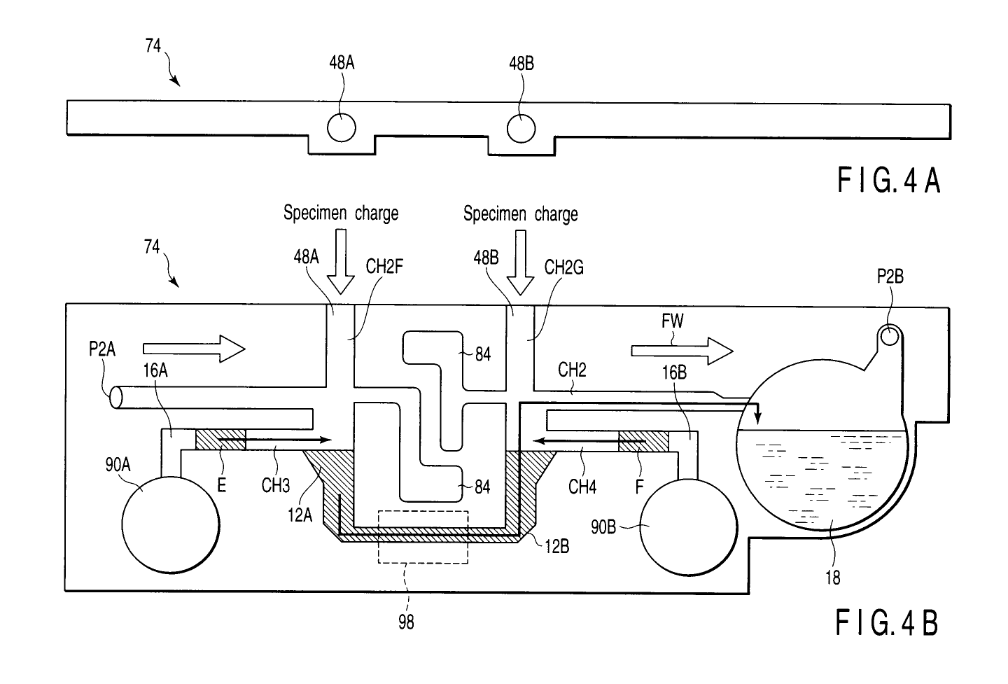

As shown in FIGS. 3D, 4A and 4B, the side structure 74 is provided with a channel structure, formed having the aforementioned channel CH2, and nucleic acid extraction/amplification chambers 12A and 12B for use as the pretreatment section 24. The structure 74 further includes reagent chambers 16A and 16B for use as the pretreatment reagent storage sections 25A and 25B, a chamber structure formed having the reagent chamber 18, and two specimen inlets 48A and 48B. Thus, the channel CH2 of the pretreatment section 24 into which the specimen S, e.g., a blood sample, is applied is provided with the nucleic acid extraction/amplification chambers 12A and 12B that communicate with the specimen inlets 48A and 48B, respectively. The chambers 12A and 12B are configured so that the specimen S can be externally applied into the channel CH2 through the specimen inlets 48A and 48B. The specimen inlets 48A and 48B are opened to the specimen S only when the specimen is applied through them and are closed after the specimen S is applied. The chambers 12A and 12B are previously stored with pretreatment reagents for extracting and amplifying the nucleic acid from the blood sample as the specimen. Further, the reagent chamber 18 contains the sample salt concentration adjustment reagent D. As shown in FIG. 1, a heating section 14 is disposed near the nucleic acid extraction/amplification chambers 12A and 12B. It can heat the specimen S (positive pretreatment reagent) in the chambers 12A and 12B. The pretreatment section 24 must be formed by deforming a member in order to control the motion of the liquid. It is formed of the rigid chip substrate with the channel CH2 or the like as grooves and an elastic body such as silicone rubber that covers the substrate surface. The covering material is not limited to silicone rubber but may alternatively be a fluoro-rubber, such as FKM or FPM, or ethylene-propylene rubber, such as EP, EPDM, or EPT. Further, the material is not limited to rubber but may be a thin film of PET, PP, PVC, or PE. If the film is used, the pretreatment section 24 has a partially deformable pouch structure instead of having the form of a rigid box.

If the specimen S is a human- or animal-derived specimen, it may be a virus, fungus, plant cell, etc., as well as blood, hair root, nail, fingerprint, oral mucosa, cell, etc. Further, these specimens may be previously subjected to a nucleic acid extraction process such as boiling when they are used.

The nucleic acid extraction/amplification chambers 12A and 12B are connected to auxiliary channels CH3 and CH4 that are different from the channel CH1. The auxiliary channels CH3 and CH4 are applied with the reagents E and F, e.g., enzymes for amplification, respectively. Push-out portions 90A and 90B are connected to the auxiliary channels CH3 and CH4, respectively. When the push-out portions 90A and 90B are actuated, in the nucleic acid detection cassette shown in FIG. 1, they are deformed to generate a pressure, whereby the reagents E and F are supplied to the first and second nucleic acid extraction/amplification chambers 12A and 12B, respectively. After the specimen (sample) S is applied into the nucleic acid extraction/amplification chambers 12A and 12B and heat-treated, the reagents E and F can be applied into the specimen chambers 12A and 12B, respectively. Thus, the respective temperatures of the specimen chambers before and after the applying of the reagents E and F can be controlled individually, so that various reaction conditions can be set.

If the amounts of the added reagents E and F are as small as about 5 to 20 μL, the reagents E and F can also be fed by means of local push mechanisms 92A and 92B, as shown in FIG. 1, without using any feed pump for the entire cassette. The push-out portions 90A and 90B are realized as a press pump mechanism that communicates with the channel CH2. The press pump mechanism has a hollow structure such that its pump is hollow inside. When circular portions that close the hollow structure are externally pushed by the push mechanisms 92A and 92B, the internal capacity of the pump section is compressed, so that the reagents E and F are individually pushed out into the channel CH2. Thereupon, the gas, e.g., air, is moved to a press pump pressure buffer 84 that communicates with the channels CH3 and CH4, and the specimen S is caused to join the heated specimen (positive pretreatment reagent) S in the nucleic acid extraction/amplification chambers 12A and 12B.

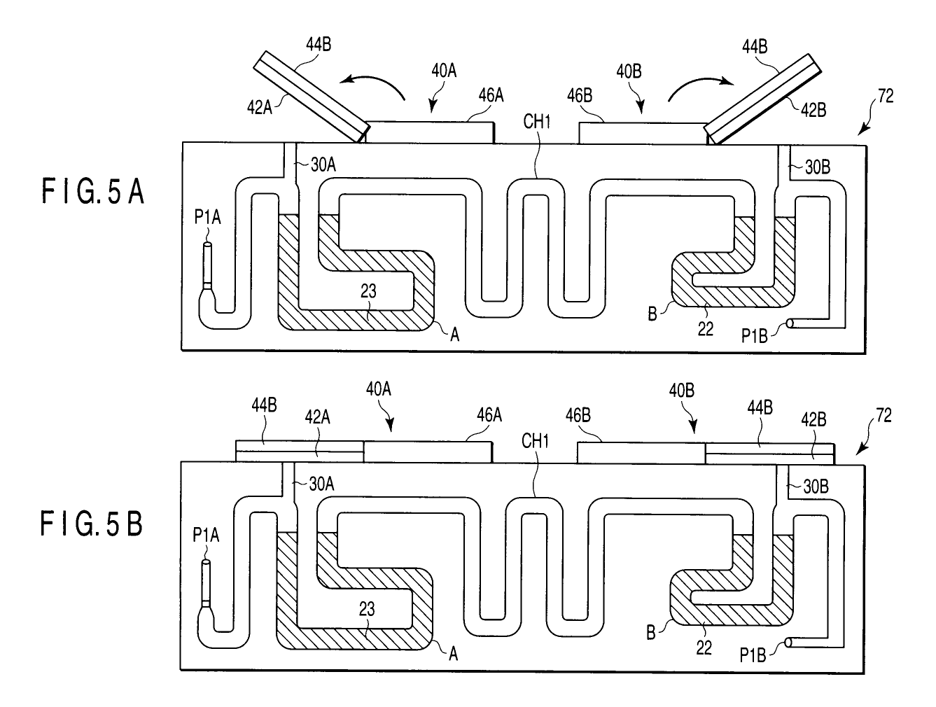

An input port P2A of the side structure 74 opens in a channel portion CH2A and is connected to the elastic piping tube 27. An output port P2B of the structure 74 opens in a channel portion CH2B and is connected to the channel CH12 of the DNA chip 10. As shown in FIGS. 5A and 5B, the side structure 72 is configured so that the washing reagent storage section 22 and the intercalator solution storage section 23 are arranged individually on its opposite sides. The washing reagent storage section 22 has a channel structure formed with the channel CH1. The channel structure contains the washing reagent B with which the nucleic acid detection substrate is washed after the hybridization reaction. Another region in the channel structure contains the intercalator solution A that interacts with the nucleic acid after the hybridization reaction. The storage section 22 is provided with gas inlet/outlet paths 30A and 30B that connect the interior of the channel CH1 and the outside of the device. Sealing mechanisms 40A and 40B are provided on those parts of the side structure 72 near the inlet/outlet paths 30A and 30B, respectively. The sealing mechanisms 40A and 40B include lid portions 44A and 44B lapped on proximal portions 46A and 46B, respectively, and adhesive members 42A and 42B are laminated to the lid portions 44A and 44B. As mentioned later, the washing reagent B and the intercalator solution A are frozen when they are kept in storage. In this frozen state, the gas inlet/outlet paths 30A and 30B are kept open. Before the nucleic acid detection device is mounted in a detection system, the frozen reagents B and A are restored to normal temperature and liquefied. After the liquefaction, the lid portions 44A and 44B are folded out from the proximal portions 46A and 46B of the sealing mechanisms 40A and 40B and stuck around the adhesive members 42A and 42B, respectively. Thus, the respective openings of the gas inlet/outlet paths 30A and 30B are covered by the lid portions 44A and 44B, as shown in FIG. 5B. In this state, the channel CH1 is fully closed and the channels CH1 and CH2 are maintained as closed channels.

Preferably, the gas inlet/outlet paths 30A and 30B should be provided in those large-capacity parts of the channel CH1 which are located as near as possible to the channel CH12 in the detection section 20 and the channel in the elastic piping tube 27. In order to minimize the length of channels in the gas inlet/outlet paths 30A and 30B, moreover, the channels 30A and 30B are made to communicate with those parts of the channel CH1 near the top surface of the side structure 72. Thus, the gas inlet/outlet paths 30A and 30B communicate with bent portions of the channel CH1 that define the washing reagent storage section 22 and the intercalator solution storage section 23, as shown in FIGS. 5A and 5B.

Preferably, the material of the reagent storage sections 22 and 23 should be deformed as the reagents A and B expand or contract. The channel CH1 and the like are formed as grooves in the rigid chip substrate, the substrate surface is covered by an elastic body such as silicone rubber, and a substrate is provided on the reverse side. The substrates are pasted together with a buffer space between them such as to absorb expansion or contraction caused by temperature changes of the reagents A and B.

The covering material is not limited to silicone rubber but may alternatively be a fluoro-rubber, such as FKM or FPM, or ethylene-propylene rubber, such as EP, EPDM, or EPT. Further, the material is not limited to rubber but may be a thin film of PET, PP, PVC, or PE. If the film is used, the pretreatment section 24 has a partially deformable pouch structure instead of having the form of a rigid box.

In the side structure 72 of the nucleic acid detection cassette, an input port P1A opens into the channel CH1 and is connected to the elastic piping tube 27 of the liquid feed section 26. An output port P1B of the structure 72 opens into the channel CH1 and is connected to the channel CH12 of the DNA chip 10. Passage sections in which the reagents A and B are stored are formed having a flow width and a cross-sectional flow area greater than those of the other channel portions.

In the side structures 72 and 74 of the nucleic acid detection cassette, the cross-sectional area of those channel portions of the channels CH1 and CH2 which extend vertically and communicate with each other is smaller than that of those channel portions which extend horizontally and communicate with each other. However, the cross-sectional area of the horizontally extending channel portions is adjusted to a size such that the channel portions can be fully closed by the surface tension of the reagent S. Thus, the reagents A, B and S can be moved through the channel portions by a pressure given to the gas by pumping operation.

The annular channels CH1 and CH2 are not uniform in cross section and each include a channel portion having a relatively large cross section and a channel portion having a relatively small cross section. The liquid specimen S and the reagents A, B, E and F never fail to cover the respective cross sections of the channels CH1 and CH2 in those parts of the channels CH1 and CH2 which have cross sections smaller than those of parts having large cross sections, depending on the shapes and surface wettability of the channels, the surface tension of the liquids A, B, S, E and F, and the viscosity and volumes of the liquid specimen S and the reagents A, B, E and F.

After the pretreatment reagent S, the nucleic acid detection substrate of the DNA chip 10, the washing reagent B, the intercalator solution A, and the sample salt concentration adjustment reagent D are incorporated into the nucleic acid detection device shown in FIGS. 1 to 5B, the device is stored frozen in a sealable bag with the gas inlet/outlet paths 30A and 30B kept open. Thus, when the device is stored frozen, the pretreatment reagents S, E and F, washing reagent B, intercalator solution A, and sample salt concentration adjustment reagent D are also frozen and prevented from moving in the channels CH1 and CH2. In nucleic acid detection, the frozen nucleic acid detection device is taken out in a laboratory at normal temperature. Then, the interior of the device is restored to normal temperature in about 30 minutes, whereupon the pretreatment reagent S, washing reagent B, intercalator solution A, and sample salt concentration adjustment reagent D are thawed. Thereafter, the device is taken out of the sealed bag, and the gas inlet/outlet paths 30A and 30B are closed by the sealing mechanisms 40A and 40B, respectively, whereupon the channels CH1 and CH2 are kept sealed. Preferably, the device should be configured so that the channels 30A and 30B can be closed by the sealing mechanisms 40A and 40B from outside the bag before the device is taken out of the bag. Thereafter, the blood sample as the specimen S is applied into the nucleic acid extraction/amplification chambers 12A and 12B of the pretreatment section 24 with the sealed state maintained. Then, the nucleic acid detection device is set in a nucleic acid detector (not shown) and used for the nucleic acid detection.

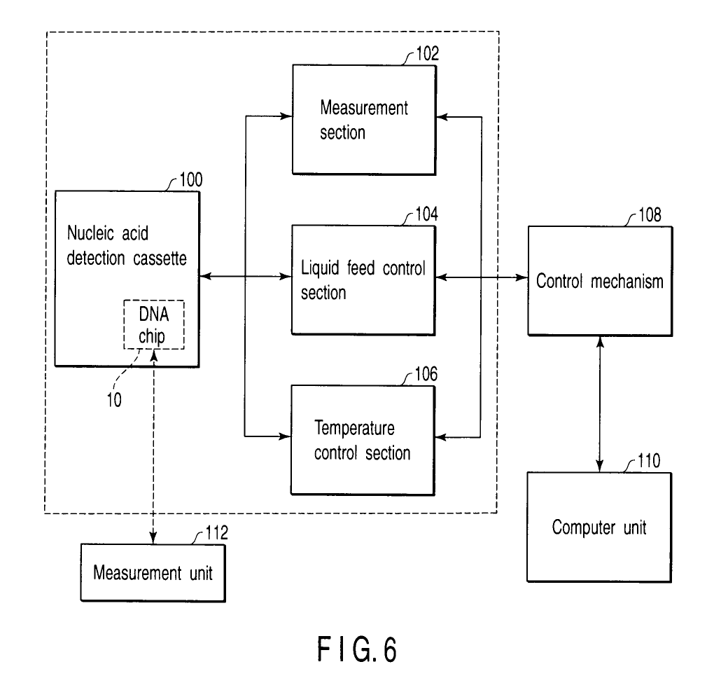

The nucleic acid detector in which the nucleic acid detection device (nucleic acid detection cassette) is incorporated for the nucleic acid detection is provided with a measurement section 102, which measures the temperatures of the reagents A and B and the sample S in a nucleic acid detection cassette 100, and a liquid feed control section 104 for controlling the feed of the reagents A and B and the sample S in the channels CH1 and CH2, as shown in FIG. 6. The control section 104 includes the pressure roller 94 and the push mechanisms 92A, 92B and 92C. Further, the nucleic acid detection device is provided with a temperature control section 106, which controls the temperatures of heating heads 14-1 and 14-2 of the heating section 14, thereby controlling the temperature of the sample S. The temperature control section 106 includes a detection section heater 98, which heats the DNA chip 10 to control the temperature of the specimen or reagent that flows through the channel CH12 therein. The measurement section 102, liquid feed control section 104, and temperature control section 106 are controlled by a control mechanism 108 under the control of a computer unit 110. The nucleic acid detection device is further provided with a measurement unit 112 for measuring reactions caused in the DNA chip 10. The measurement unit 112 utilizes the electrode connector 92 shown in FIG. 1 to detect a detection signal from the electrode pads 10C and determine the electrode 10B for conduction, thereby specifying the nucleic acid.

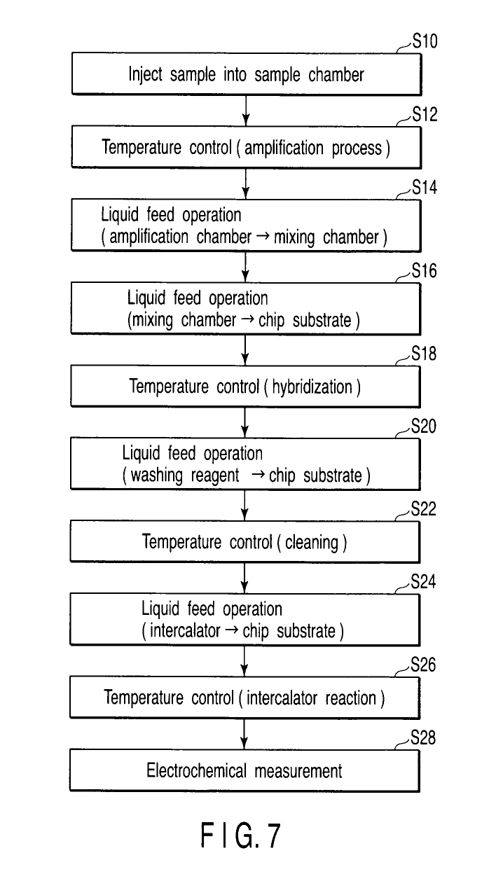

In this nucleic acid detection device, the nucleic acid detection is performed in the following steps of procedure shown in FIG. 7 (see FIGS. 1 to 3).

First, sampling rods 60 are inserted individually into the inlets 48A and 48B, whereby the specimen S is mixed into the pretreatment reagent (Step S10). At this point in time, the gas inlet/outlet paths 30A and 30B are already closed by the sealing mechanisms 40A and 40B, respectively. Then, the detection device is mounted in the nucleic acid detector, and the specimen S in the pretreatment reagent is heated by the heating section 14 so that the target DNA is amplified (Step S12). As the specimen S in the reagent is heated at, for example, 95° C. for 5 minutes or more by the heating heads 14-1 and 14-2 of the heating section 14, the nucleic acid in the specimen S is extracted. A reagent (enzyme) that is not heat-resistant, e.g., a LAMP amplification enzyme, is stored in the storage sections 25A and 25B of the channel CH2 at a distance from the heating region, as shown in FIG. 3. After the heat treatment is finished, the heated specimen S is doped with a reagent.

Thereafter, the pressure roller 94 is actuated so that a sample solution that contains the amplified specimen S is fed in the forward direction FW to the sample storage chamber 18 through the channel CH2 (Step S14). Thus, the gas in the channel CH2 is pushed by a push force from the pump, whereupon the specimen S is fed into the reagent chamber 18 and mixed with the sample salt concentration adjustment reagent D. Thereafter, the push mechanism 92C is actuated so that an output port of the chamber 18 that communicates with the DNA chip 10 is closed by the adjustment reagent D that is mixed with a reagent. In this state, the pump is actuated so that the mixed specimen S is fed to the DNA chip 10 (Step S16). Thus, in the DNA chip 10, the target DNA is hybridized with the probe DNAs under temperature control (Step S18). Then, the pressure roller 94 is reversely operated so that the sample solution in the DNA chip 10 is returned from it to the sample storage chamber 18, and the reagent B (cleaning solution) is fed to the channel CH12 in the DNA chip 10 (Step S20). Specifically, the pump is operated in the backward direction BW so that the reagent B is pushed and fed into the DNA chip 10 by the reagent A in the channel CH1 and the gas 16. Most of the specimen (DNA sample) S is pushed out into the channel CH2 outside the chip 10 by a pressure generated as the reagent B flows in. As the reagent B is supplied to the DNA chip 10 kept at a predetermined temperature, all the DNAs except the target DNA that is hybridized to the probe DNAs of the chip 10 and has a sequence complementary to those of the probe DNAs are washed with the reagent B (Step S22). When the pressure roller 94, that is, the pump, further continues to be operated in the backward direction BW, the reagent A (intercalator solution) additionally pushes the gas so that the reagent B mixed with all the DNAs to be cleaned except the target DNA is pushed out into the channel CH2 outside the DNA chip 10.

As the pump continues to operate, the reagent A is fed to the channel CH12 in the DNA chip 10 under the gas pressure (Step S24). In the DNA chip 10, molecules of the intercalator solution are added to a combination of the probe DNAs of the chip 10 and the target DNA that are hybridized together, whereupon a reaction in the intercalator solution is caused in the chip 10 (Step S26). Thus, an oxidation/reduction current that is generated by the application of voltage between the individual electrodes and the counter electrodes is detected in the individual electrodes with the intercalator solution injected. Thereupon, an electrochemical reaction is determined to specify the sample nucleic acid. Since the base sequence of the probe DNAs of the DNA chip 10 is generally known, that of the target DNA can be determined by specifying the individual electrodes in which a large value is detected for the oxidation/reduction current. Thus, it is revealed that the base sequence in a to-be-detected region of the target DNA is complementary to the probe DNA sequence of the current detection electrodes (Step S28).

Although the present embodiment is applied to the electrochemical nucleic acid detection method using the intercalator, the method of nucleic acid detection according to the present invention is not limited in particular, but may alternatively be an electric or optical method. Some detection methods in which no intercalator is used may dispense with the intercalator solution storage section 23.

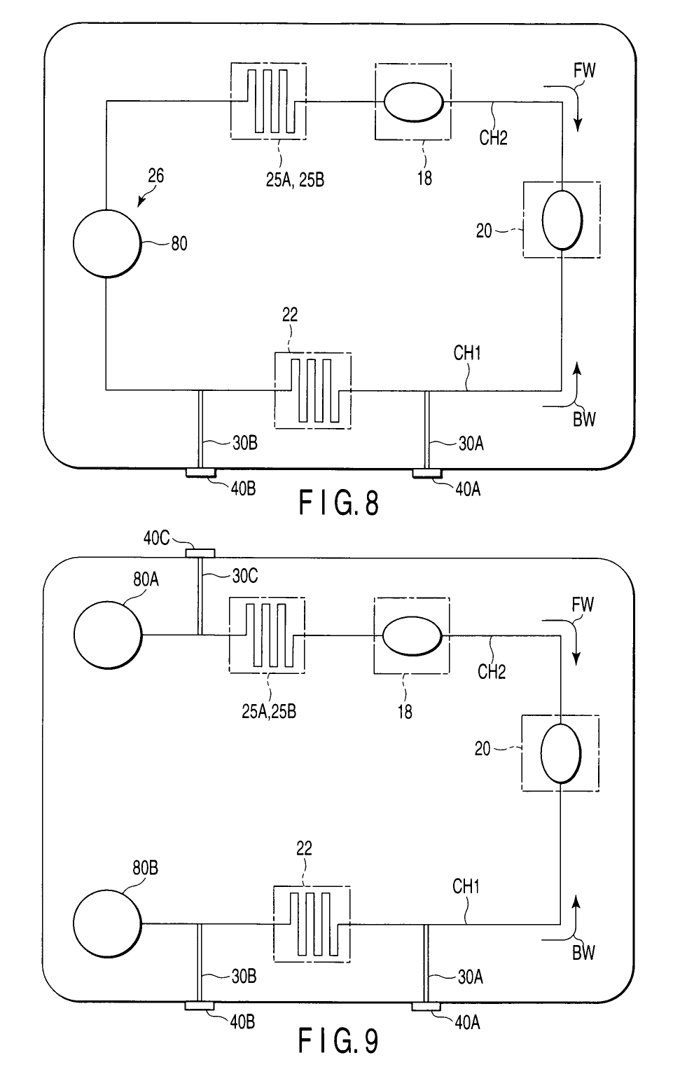

FIG. 8 diagrammatically illustrates the nucleic acid detection device shown in FIGS. 1 to 3D. It is to be noted that the intercalator solution storage section 23 and the like are not shown in the diagram of FIG. 8 for simplicity.

As mentioned before, the pretreatment reagent storage sections 25A and 25B, reagent chamber 18, detection section 20, washing reagent storage section 22, and liquid feed section 26 (pump 80) are connected in a ring by the channels CH1 and CH2. The gas inlet/outlet paths 30A and 30B are configured to be coupled to channels on the opposite sides of the storage section 22. In the nucleic acid detection device constructed in this manner, the pretreatment reagents S, E and F, the washing reagent B, and the like are expanded as they are frozen. Changes of pressure in the channels CH1 and CH2 that are caused by the expansion of the reagents are eased by the gas inlet/outlet paths 30A and 30B that are open. As the reagents S, E, F and B and the like are frozen, moreover, they can be fixed in the nucleic acid detection device so as to be prevented from moving in the channels CH1 and CH2. Thus, the detection device can be conveyed with ease, and the pretreatment reagents S, E and F, the washing reagent B, and the like can be prevented from being mixed together and rendering the device unusable. Even if the reagents S, E, F and B and the like are frozen, furthermore, they evaporate slightly. Pressure changes in the channels CH1 and CH2 that are caused by the evaporated gas are eased by the gas inlet/outlet paths 30A and 30B that are open.

In the nucleic acid detection using the nucleic acid detection device, the device that is stored frozen is taken out in the normal-temperature laboratory and its interior is restored to normal temperature in about 30 minutes, whereupon the pretreatment reagent S, washing reagent B, intercalator solution A, and sample salt concentration adjustment reagent D are thawed. In this thawing process, pressure changes in the channels CH1 and CH2 can also be eased by the gas inlet/outlet paths 30A and 30B that are open.

After the pretreatment reagent S, washing reagent B, intercalator solution A, and sample salt concentration adjustment reagent D are liquefied, the gas inlet/outlet paths 30A and 30B are closed by the sealing mechanisms 40A and 40B, respectively, so that the channels CH1 and CH2 can be kept securely sealed. Thus, the pretreatment reagent S, washing reagent B, intercalator solution A, and sample salt concentration adjustment reagent D can be securely moved in the channels CH1 and CH2 by the pressure roller 94 and the push mechanisms 92A, 92B and 92C.

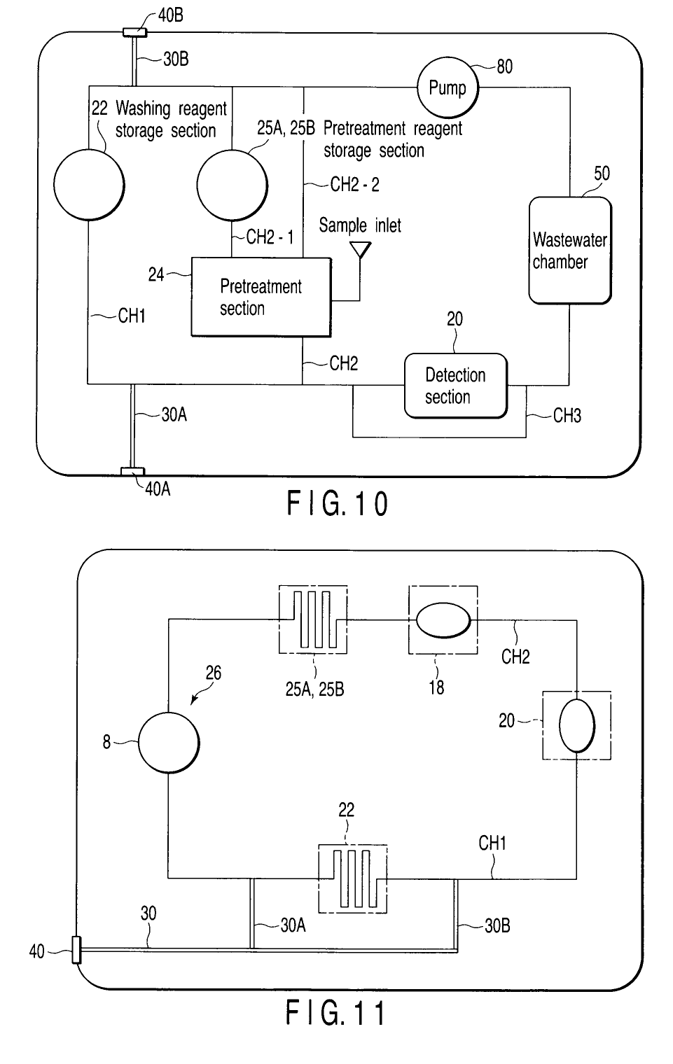

Although the channels CH1 and CH2 shown in FIG. 8 are formed in a ring, their shape is not limited to the ring shape, but pump sections, e.g., push pumps 80A and 80B, may be provided individually at the opposite ends of the channels CH1 and CH2, as shown in FIG. 9. As shown in FIG. 9, moreover, the side structure 74 may be provided with a gas inlet/outlet path 30C, which extends from the channel CH1, as well as from the channel CH2, and communicate with the outside, and a sealing mechanism 40C. Preferably, the gas inlet/outlet path 30C should be made to communicate with that part of the channel CH1 which is situated close to the pretreatment reagent storage sections 25A and 25B. As shown in FIG. 10, furthermore, the channel CH2 may be diverged into two channels CH2-1 and CH2-2, which are connected to the pretreatment section 24. In this case, the channel CH2-1 is provided with the pretreatment reagent storage sections 25A and 25B, the channels CH1 and CH2 are joined together and connected to the detection section 20, and the channels CH2-1 and CH2-2 and the channel CH1 are also joined together and connected to the pump 80. Further, the pretreatment section 24 may be provided with the inlets 48A and 48B for the sample S. In this case, the detection section 20 is provided with a bypass channel CH3, and a wastewater chamber 50 is provided between the detection section 20 and the pump 80. The nucleic acid detection device in which the channels CH1 and CH2 are arranged in parallel with each other may also be configured so that the gas inlet/outlet paths 30A and 30B communicate with the channel CH2 on either side of the washing reagent storage section 22 and can be closed by the sealing mechanisms 40A and 40B, respectively. The nucleic acid detection device shown in FIG. 10 may be configured so that pressure from the pump 80 is alternatively applied to each of the channels CH1-1, CH1-2, CH2 and CH3, whereby the specimen S is mixed into the pretreatment reagent and supplied to the detection section 20, and thereafter, the washing reagent B is supplied to the detection section 20 for nucleic acid detection.

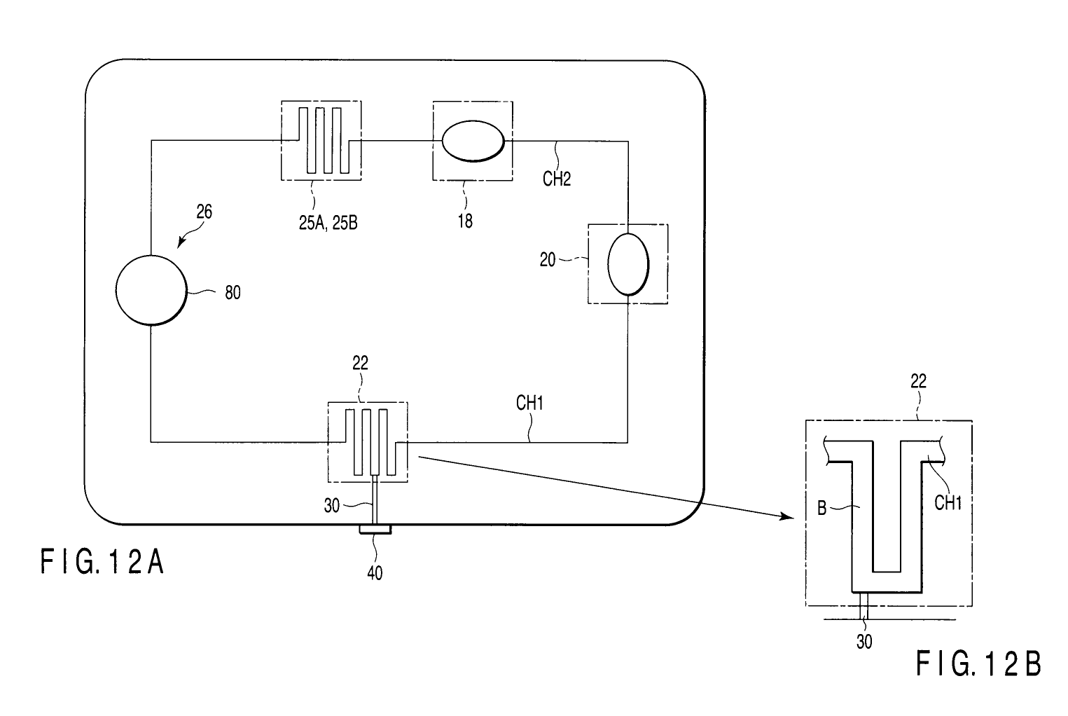

As shown in FIG. 11, moreover, the gas inlet/outlet paths 30A and 30B are coupled to the channels on the opposite sides of the washing reagent storage section 22. Alternatively, however, these two channels may be joined to one inlet/outlet path 30, which opens on the side structure 72. Since the gas inlet/outlet paths 30A and 30B open in one spot in this structure, only one sealing mechanism 40 is essential, so that the external structure of the nucleic acid detection device can be simplified. The divergence of the gas inlet/outlet paths 30A and 30B is not limited to the structure shown in FIG. 11. Alternatively, the inlet/outlet paths may be further diverged so that they are connected in any other places than the channels on the opposite sides of the storage section 22. As shown in FIGS. 12A and 12B, the gas inlet/outlet path 30 may be made to communicate directly with the washing reagent storage section 22. In the nucleic acid detection device having this structure, the gas inlet/outlet path 30 can be used as an inlet through which the washing reagent B is injected into the device. If the reagent B is injected into the device, furthermore, a gas in the channel with the same volume as the reagent must be discharged from the device. An exhaust port for the air discharge can also be used as the gas inlet/outlet path 30. Various configurations may be employed for the nucleic acid detection device and the gas inlet/outlet paths.

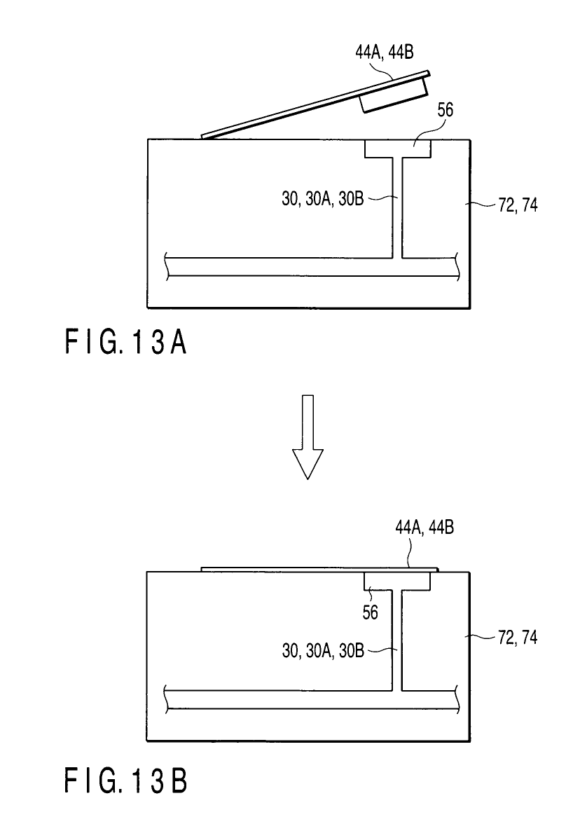

As shown in FIGS. 13A and 13B, the gas inlet/outlet path 30, 30A or 30B may be formed with various shapes. Specifically, a recess 56 may be formed in the top surface of the side structure 72 or 74 such that the inlet/outlet path 30, 30A or 30B opens therein. Further, each sealing mechanism 40 is not limited to the structure formed of the adhesive member 42A or 42B disposed on the lid portion 44A or 44B. Alternatively, as shown in FIGS. 13A and 13B, each lid portion 44A or 44B may be formed of an elastic member itself such that it can be fitted in the recess 56 to close the gas inlet/outlet path 30, 30A or 30B.

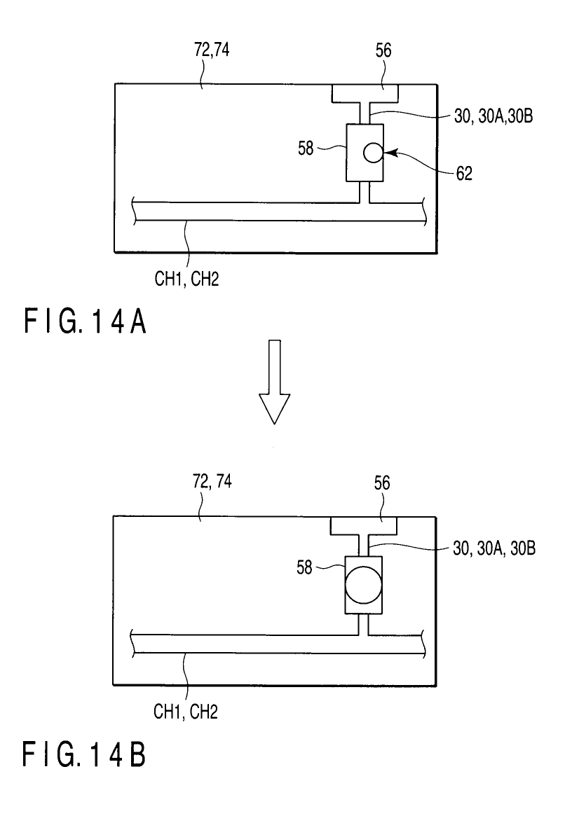

As shown in FIGS. 14A and 14B, moreover, an expansion chamber 58 may be provided in the gas inlet/outlet path 30, 30A or 30B. In this case, the expansion chamber 58 contains therein a stopper member 62 that can be expanded by heat or an electrical signal applied from the outside. When the nucleic acid detection device is stored frozen, as shown in FIG. 14A, the stopper member 62 is kept contracted, and the channel CH1 or CH2 communicates with the outside through the inlet/outlet path 30, 30A or 30B. Before the nucleic acid is detected by the detection device, on the other hand, the stopper member 62 is heated or supplied with current or voltage and expanded, as shown in FIG. 14B. In consequence, the inlet/outlet path 30, 30A or 30B is closed by the stopper member 62. Thereupon, the channel CH1 or CH2 is kept closed. The stopper member 62 may also be made of a material such that it is contracted again when it is cooled or supplied with a reverse voltage or current.

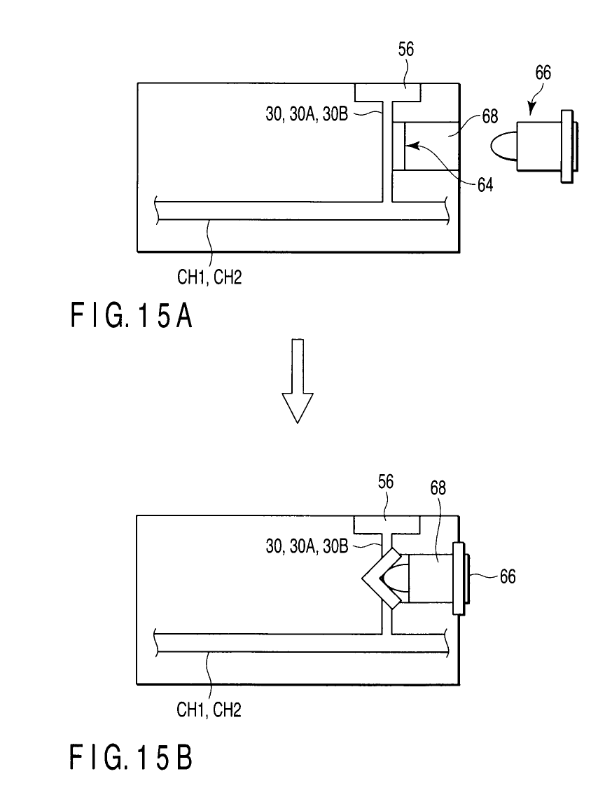

As shown in FIGS. 15A and 15B, a wall surface that defines each gas inlet/outlet path 30, 30A or 30B is formed of an elastic or easily breakable member 64. The side structure 72 or 74 is provided with an approach channel 68 for a sealing pin 66 that is externally guided to the member 64. Before the nucleic acid is detected by the nucleic acid detection device, the sealing pin 66 may be introduced into the approach channel 68 so that the member 64 is broken by the sealing pin 66 to close the gas inlet/outlet path 30, 30A or 30B, as shown in FIG. 15B.

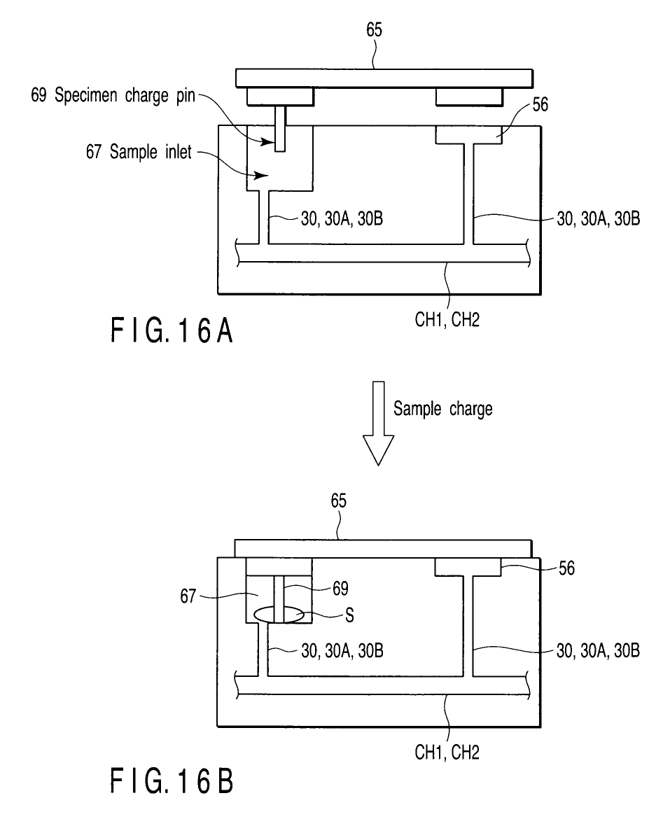

As shown in FIGS. 16A and 16B, the gas inlet/outlet path 30, 30A or 30B may be made to communicate with an open chamber 67 for sample charge so that the chamber 67 can be closed by a stopper member 65 that has a specimen charge pin 69. When the sample (specimen) S is applied, it may be attached to the distal end of the pin 69, which can be inserted into the open chamber 67 so that the chamber 67 is closed by the stopper member 65. The gas inlet/outlet path 30, 30A or 30B is closed by the stopper member 65 in a manner such that the stopper member 65 is fitted into the chamber 67 and the recess 56 to close them.

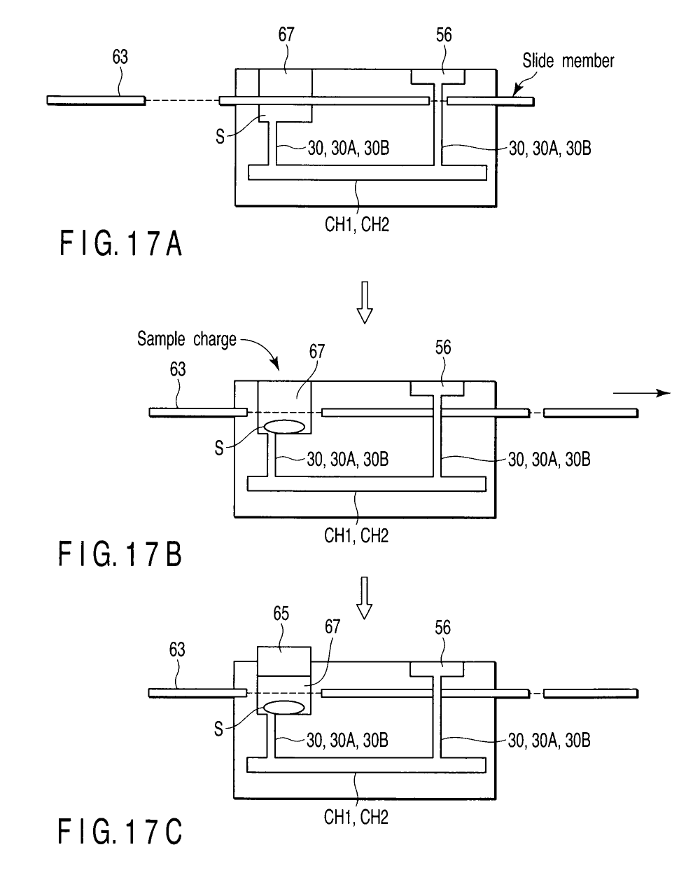

In an arrangement shown in FIGS. 17A, 17B and 17C, a slide member 63 is slidably disposed in the side structure 72 or 74. As shown in FIG. 17A, an opening of the slide member 63 is situated in the gas inlet/outlet path 30, 30A or 30B so that the channel CH1 or CH12 is opened through the channel 30, 30A or 30B. When the sample S is applied, as shown in FIG. 17B, the slide member 63 is slid so that its opening is situated in the chamber 67 to allow the chamber 67 to open, whereupon the channel 30, 30A or 30B is closed by the slide member 63. If the sample is applied into the chamber 67, the chamber 67 is closed by the stopper member 65, whereupon the channel CH1 or CH12 is closed against the outside.

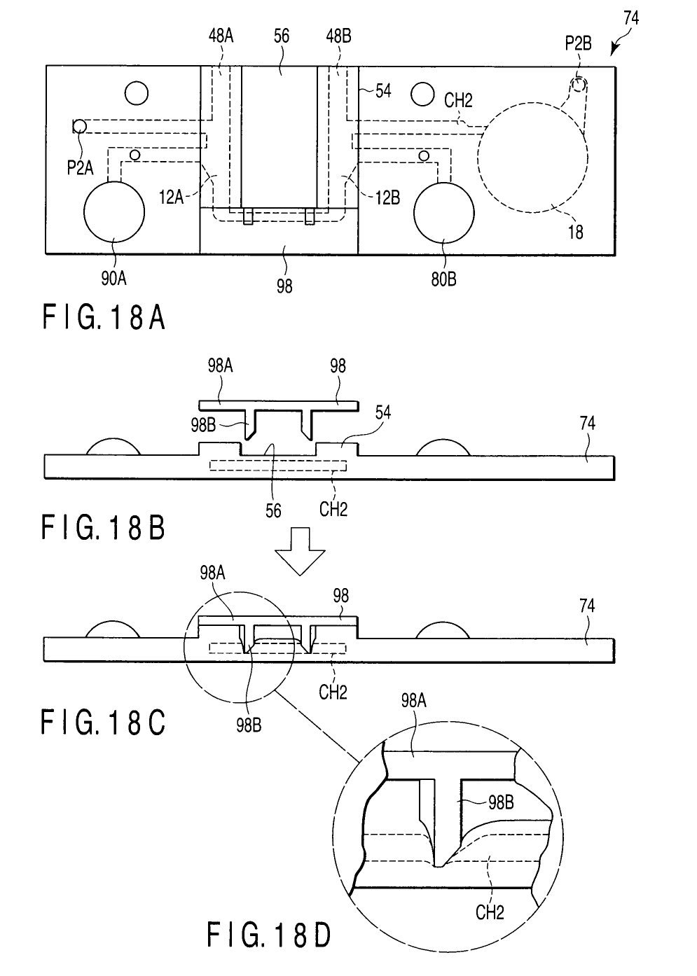

In the cassette structure having a plurality of specimen inlets 48A and 48B, those parts of the channel CH2 that communicate with the inlets 48A and 48B may be clamped (tightened and fixed) by means of a clip 98 for use as a fastener, as shown in FIGS. 18A, 18B and 18C. This is intended to prevent the reagent from unexpectedly moving in the cassette being transported, thereby preventing the internal pressure from being unbalanced and causing the reagent to move unexpectedly during the charge of the sample S.

More specifically, as shown in FIG. 18A, a clip attachment portion 52 for channel sealing is provided on the outer surface of the side structure 74 so as to face the specimen inlets 48A and 48B and the nucleic acid extraction/amplification chambers 12A and 12B. The attachment portion 52 is composed of pedestal portions 54, which are formed on the outer surface of the side structure 74 and extend along the inlets 48A and 48B, and the recess 56 formed between the pedestal portions 54. As shown in FIGS. 18B and 18C, moreover, the clip 98 that is attached to the attachment portion 52 is composed of blade portions 98B protruding from a blade-shaped proximal portion 98A. The blade portions 98B are spaced so as to fit the recess 56. The clip 98 for channel sealing is fixedly mounted on the attachment portion 52 in a manner such that the blade portions 98B are forcedly fitted into the recess 56, resisting the repulsive force of an elastic material that forms the side structure 74, as shown in FIGS. 18B and 18C. When the clip 98 is set on the attachment portion 52, the blade portions 98B are pushed in an elastic area that defines the channel CH2 between the nucleic acid extraction/amplification chambers 12A and 12B, as shown in FIGS. 18C and 18D. Thus, the peripheral wall of the channel CH2 between the chambers 12A and 12B is crushed so that the channel CH2 is blocked.

The channel sealing clip 98 is attached to the attachment portion 52 after the detection device is prepared. Thus, the channel CH2 between the extraction/amplification chambers 12A and 12B is blocked by the blade portions 98B. Thereafter, different reagents are injected individually into the chambers 12A and 12B and stored therein. These two chambers 12A and 12B communicate with each other by means of the channel CH2. Since the channel CH2 is blocked by the blade portions 98B, however, the reagents can be prevented from being unexpectedly mixed with each other. When the different reagents are thus held individually in the extraction/amplification chambers 12A and 12B, the detection device and the liquids therein are frozen and delivered in this state to a user.

When the nucleic acid detection device in the frozen state is delivered to the user, it is thawed and prepared for nucleic acid detection. When the preparation is completed, the specimen inlets 48A and 48B are opened first. When the sampling rods (not shown) to which the specimen adheres are inserted individually into the inlets 48A and 48B, the inlets are blocked at once. When the specimen is applied, the channel between the nucleic acid extraction/amplification chambers 12A and 12B is blocked by the channel sealing clip 98. Therefore, the reagents in the chambers 12A and 12B can be prevented from moving to wrong positions. Thereafter, the clip 98 is removed, the nucleic acid detection device is attached to the detector, and the aforementioned nucleic acid detection is started.

In the detection process described above, the specimen S is externally heated by the heating section 14. In order to restrict the object of heating in this process, the region where the channel CH2 is closed by the heating section 14 so that the specimen is heated by the peripheral communicating channel may be restricted.

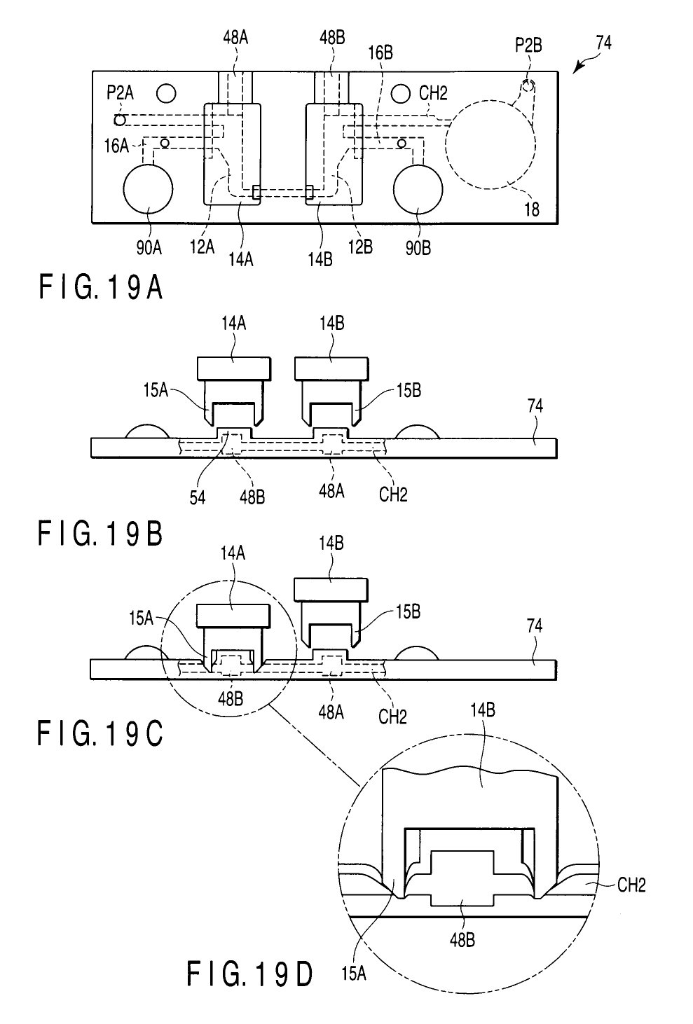

More specifically, as shown in FIGS. 19A, 19B, 19C and 19D, the heating section 14 is provided with heating heads 14A and 14B, which individually contain heaters corresponding to the extraction/amplification chambers 12A and 12B. Blades 15A and 15B are provided on the distal end of each of the heating heads 14A and 14B. The blades 15A and 15B have a shape such that they can nip each corresponding one of the pedestal portions 54 that extend on the outer surface of the side structure 74.

In the extraction/amplification process, the heating section 14 is located opposite the side structure 74, as shown in FIGS. 19A and 19B, and the respective distal ends of the heating heads 14A and 14B are pressed against their corresponding pedestal portions 54. As shown in FIGS. 19C and 19D, the blades 15A and 15B are brought into contact with the regions on the opposite sides of each pedestal portion 54 and deform those regions as they are pressed against the elastic side structure 74. Thereupon, the regions in contact with the blades 15A and 15B are deformed to block the channel CH2 under their surfaces. Thus, the nucleic acid extraction/amplification chambers 12A and 12B in the regions opposite to the heating heads 14A and 14B are isolated from the channel CH2. Since the heating heads 14A and 14B can be advanced and retreated independently, the chambers 12A and 12B can be individually isolated from the channel CH2. By pressing the heads 14A and 14B individually against the chambers 12A and 12B, therefore, the chambers 12A and 12B can be individually heated, so that reactions of the nucleic acid therein can be prompted independently.

The nucleic acid extraction/amplification chambers 12A and 12B are made to communicate with each other by their adjacent regions and the channel CH2. When the chambers 12A and 12B are heated, there is a possibility of the solution being evaporated and dispersed to other regions, thereby hindering satisfactory control of the reactions. Since the blades 15A and 15B block the channel around them, as mentioned before, the chambers 12A and 12B to be heated can be spatially isolated from their surroundings. Besides, the solution in the chambers can be prevented from evaporated and dispersed by heating. Thus, the extraction/amplification process can be performed with good controllability.

According to the present invention, as described above, the nucleic acid detection device having the structure for preventing mixing-in of undetectable nucleic acid molecules and leakage of nucleic acid samples to the outside can be stored for a long period of time. If the gas is contracted in the channel by a temperature drop, the internal pressure can be kept constant by introducing the gas outside the device through the gas inlet/outlet path, whereby the movement of the reagent can be restrained. If the temperature rises, on the other hand, the gas can be discharged through the gas inlet/outlet path.

In the case where the gas inlet/outlet paths are set in the pretreatment reagent storage sections and their neighboring pretreatment section, moreover, evaporation of an infinitesimal amount of the pretreatment reagent has a large influence. However, this influence can be suppressed by setting the gas inlet/outlet paths in any other regions than the pretreatment reagent storage sections and their neighboring pretreatment section. Possibly, furthermore, the undetectable nucleic acid molecules may be mixed in through these gas inlet/outlet paths. If they get into the pretreatment section and the pretreatment reagent storage section, however, the undetectable nucleic acid molecules are inevitably amplified, resulting in a very fatal outcome. If the undetectable nucleic acid molecules get into any other places than the pretreatment section and the pretreatment reagent storage section, they cannot be amplified, so that the reliability is improved.

### EXAMPLE 1

A usage example of the fully automated nucleic acid detection device according to the foregoing embodiment will now be described in detail.

1. Preparation for Nucleic Acid Detection Device

The following reagents were prepared to be set in the nucleic acid detection device.

Pretreatment reagent 1: LAMP amplification buffer, primer

Pretreatment reagent 2: LAMP amplification enzyme

Cleaning reagent: SSC

Intercalator solution: Hoechst 33258

Further, a nucleic acid detection substrate with a plurality of electrodes was prepared in which nucleic acid probes having the following sequences were immobilized on each electrode.

| | |
|---|---|
| Nucleic acid probe A: | ATGCTTTCCGTGGCA |
| Nucleic acid probe B: | ATGCTTTGCGTGGCA |

The pretreatment reagents 1 and 2 were set in the pretreatment reagent storage section, the washing reagent and the intercalator solution in the washing reagent storage section, and the nucleic acid detection substrate in the detection section.

The gas inlet/outlet paths are kept open.

2. Storage of Nucleic Acid Detection Device

The various reagents are encapsulated and put into the bag, which is sealed with the gas inlet/outlet paths open. The device is left to stand in a freezer that is kept at −20° C.

3. Fully Automated Nucleic Acid Detection

Nucleic acid detection is performed after one month of frozen storage.

The nucleic acid detection device is taken out of the freezer and left to stand in a normal-temperature room for about 30 minutes, whereby the entire device is restored to normal temperature. The device is taken out of the sealed bag, and the gas inlet/outlet paths are sealed by means of the sealing mechanisms provided with the adhesive members. Thereafter, the blood sample to be detected is applied through the sample inlets in the pretreatment section, and the sample inlets are closed. The nucleic acid detection device is set in the nucleic acid detector. The nucleic acid detector is provided with a temperature control mechanism, liquid feed mechanism, detection mechanism, and signal analysis mechanism.

First, the pretreatment section that contains the pretreatment reagent mixed with the blood sample is kept at 95° C. for 5 minutes, whereby a nucleic acid extraction reaction is performed. Then, the pretreatment reagent that contains the enzyme is fed to the pretreatment section and mixed. Subsequently, an amplification reaction is performed by keeping the pretreatment section at 60° C. for 60 minutes.

The amplified sample is fed to the area of the chamber structure of the pretreatment section and mixed with the sample salt concentration adjustment reagent. Thereafter, the sample is fed to the detection section, which is kept at 57° C. for 20 minutes for the hybridization reaction. Then, the washing reagent is fed to the detection section and kept at 48° C. for 20 minutes, whereby a cleaning reaction is performed such that the nonspecifically adsorbed nucleic acid is removed. Further, the intercalator solution is fed to the detection section, and the hybridized nucleic acid molecules and intercalator solution molecules are caused to interact with one another. Finally, a voltage is applied to each electrode, the current value obtained by an oxidation reaction of the intercalator solution molecules is determined, and nucleic acid detection is performed by analyzing the obtained current signal.

Since the current value obtained from the electrode on which the nucleic acid probe A was immobilized was larger than the current value obtained from the electrode on which the nucleic acid probe B was immobilized, the DNA in the sampled specimen was found to have a sequence CTG CCACGGAAAG CAT.

According to the present invention, the nucleic acid detection device having the structure for preventing mixing-in of undetectable nucleic acid molecules and leakage of nucleic acid samples to the outside can be stored for a long period of time. If the gas is contracted in the channel by a temperature drop, the internal pressure can be kept constant by introducing the gas outside the device through the gas inlet/outlet path, whereby the movement of the reagent can be restrained. If the temperature rises, on the other hand, the gas can be discharged through the gas inlet/outlet path.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A nucleic acid detection device comprising:
a pretreatment section configured to treat a nucleic acid contained in a specimen before nucleic acid detection;
a detection section configured to detect the pretreated nucleic acid;
a pretreatment reagent storage section which communicates with the pretreatment section and stores a pretreatment reagent to be used for the treatment in the pretreatment section;
a washing reagent storage section configured to store a washing reagent which is used to wash the detection section after the specimen is supplied from the pretreatment section to the detection section;
a channel including a first channel portion through which the washing reagent storage section communicates with the detection section and a second channel portion through which the pretreatment section communicates with the detection section, the first and second channel portions being able to form a closed channel;
a gas inlet/outlet path through which the closed channel communicates with the outside; and
a sealing mechanism which blocks the gas inlet/outlet path and keeps the channel closed before the nucleic acid detection, the sealing mechanism being configured to keep the gas inlet/outlet path and the channel open when the pretreatment reagent and the washing reagent are stored frozen and externally block the gas inlet/outlet path after the pretreatment reagent and the washing reagent are thawed.

2. A nucleic acid detection device according to claim 1, wherein pretreatment in the pretreatment section includes a nucleic acid extraction process for extracting the nucleic acid and/or a nucleic acid amplification process of amplifying the nucleic acid.

3. A nucleic acid detection device according to claim 1, wherein the gas inlet/outlet path communicates with the channel on either side of the washing reagent storage section.

4. A nucleic acid detection device according to claim 1, further comprising a first plate section formed with the washing reagent storage section and the first channel portion, a second plate section formed with the pretreatment section, the pretreatment reagent storage section, and the second channel portion, and a third plate section provided between the first and second plate sections and formed with the detection section.

5. A nucleic acid detection device according to claim 1, wherein the gas inlet/outlet path is formed in the first plate section and communicates with the first channel portion.

6. A nucleic acid detection device according to claim 1, wherein the sealing mechanism is provided with a stopper structure which closes an opening of the gas inlet/outlet path, and at least one of the gas inlet/outlet path and the stopper structure is formed of an elastic member.

7. A nucleic acid detection device according to claim 6, wherein the sealing mechanism is provided integrally with a specimen applying device for applying the specimen into the pretreatment section.

8. A nucleic acid detection device according to claim 1, wherein the sealing mechanism includes a member which expands or contracts depending on a temperature and is configured to block the gas inlet/outlet path when the member is expanded.

9. A nucleic acid detection device according to claim 1, wherein the sealing mechanism includes a member which expands or contracts in response to an electrical signal given from the outside and is configured to block the gas inlet/outlet path when the member is expanded.

10. A nucleic acid detection device according to claim 1, wherein the gas inlet/outlet path functions as an inlet port through which the washing reagent is injected into the washing reagent storage section.

11. A nucleic acid detection device according to claim 1, wherein the pretreatment reagent and the washing reagent are separated from each other by a gas.

12. A nucleic acid detection device comprising:
a pretreatment section configured to treat a nucleic acid contained in a specimen before nucleic acid detection;
a detection section configured to detect the nucleic acid;
a pretreatment reagent storage section which communicates with the pretreatment section and stores a pretreatment reagent to be used for the treatment in the pretreatment section;
a washing reagent storage section configured to store a washing reagent which is used to wash the detection section after the specimen is supplied from the pretreatment section to the detection section;
a channel including a first channel portion through which the washing reagent storage section communicates with the detection section and a second channel portion through which the pretreatment section communicates with the detection section, the first and second channel portions forming a closed channel; and
a fastener which blocks a part of a channel in the pretreatment section from the channel, thereby isolating the part of the channel in the pretreatment section, the fastener having an opening function to cause the pretreatment section to communicate with the channel during the nucleic acid detection.

13. A nucleic acid detection device according to claim 12, which further comprises a gas inlet/outlet path through which the closed channel communicates with the outside and a sealing mechanism which blocks the gas inlet/outlet path and keeps the channel closed before the nucleic acid detection, the sealing mechanism being configured to keep the gas inlet/outlet path and the channel open when the pretreatment reagent and the washing reagent are stored frozen and externally block the gas inlet/outlet path after the pretreatment reagent and the washing reagent are thawed.

14. A nucleic acid detector for nucleic acid detection, which incorporates therein a nucleic acid detection device comprising a pretreatment section configured to treat a nucleic acid contained in a specimen before nucleic acid detection, a detection section configured to detect the nucleic acid, a pretreatment reagent storage section which communicates with the pretreatment section and stores a pretreatment reagent to be used for the treatment in the pretreatment section, a washing reagent storage section configured to store a washing reagent which is used to wash the detection section after the specimen is supplied from the pretreatment section to the detection section, and a channel composed of a first channel portion through which the washing reagent storage section communicates with the detection section and a second channel portion through which the pretreatment section communicates with the detection section, the first and second channel portions forming a closed channel, the nucleic acid detector comprising a heating section which blocks the pretreatment section from the channel during the nucleic acid detection and heats the pretreatment section.
